(19) United States
(12) Patent Application Publication
(10) Pub. No.: US 2025/0017773 A1
(43) Pub. Date: Jan. 16, 2025

(54) Surgical Apparatus

(71) Applicant: Nallakrishnan Family Trust, Willowbrook, IL (US)

(72) Inventor: Ravi Nallakrishnan, Willowbrook, IL (US)

(21) Appl. No.: 18/713,073

(22) PCT Filed: Nov. 29, 2022
(86) PCT No.: PCT/US2022/051145

§ 371 Date: May 23, 2024

Related U.S. Application Data
(60) Provisional application No. 63/286,726, filed on Dec. 7, 2021.

(51) Int. Cl. A61F 9/007

(52) U.S. Cl. CPC A61F 9/00745

(57) ABSTRACT

A surgical apparatus (10, 10A, 10B, 10C) includes a hollow cannula (20, 20A, 20B, 20C) including least one locking aperture (34, 34A, 34B, 34C) extending therein. The apparatus (10, 10A, 10B, 10C) has a hollow tip (40, 40A, 40B, 40C, 40D, 40E, 40F, 40G, 40H, 40I) with a base portion (42, 42B, 42C, 42D, 42E, 42F, 42G, 42H, 42I) including at least one projection (50, 50A, 50B, 50C, 50D, 50E, 50F, 50G, 50H, 50I) extending within the at least one locking aperture (34, 34A, 34B, 34C) of the cannula (20, 20A, 20B, 20C) for maintaining the tip (40, 40A, 40B, 40C, 40D, 40E, 40F, 40G, 40H, 40I) in position. The tip (40, 40A, 40B, 40C, 40D, 40E, 40F, 40G, 40H, 40I) has an exposed end portion (44, 44A, 44B, 44C, 44D, 44E, 44F, 44G, 44H, 44I) extending beyond the cannula (20, 20A, 20B, 20C) and defines at least one port (48, 48A, 48B, 48C, 48D, 48E, 48F, 48G, 48H, 48I) in fluid communication with the interior of the cannula (20, 20A, 20B, 20C).

### PRIORITY

This application claims priority of U.S. Provisional Patent Application Ser. No. 63/286,726, filed Dec. 7, 2021, the entire contents of which is incorporated herein by reference.

### FIELD OF THE INVENTION

The present invention relates generally to surgical instruments used in opthalmological surgery and, more particularly, to improved irrigation and aspiration instruments.

### BACKGROUND OF THE INVENTION

Phacoemulsification has come to be a technique of choice for the removal of damaged or diseased lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. The lens of the eye is held within a capsular bag positioned behind the iris in the anterior chamber of the eye. When the lens becomes damaged or diseased, a common surgical technique is to remove the lens and replace it with an artificial intraocular lens (IOL). Removal of the lens is commonly carried out by physical dismemberment of the lens and phacoemulsification, that is, using an instrument to which vibrational energy is transmitted by a phacoemulsification hand piece.

During phacoemulsification, the lens is broken into fragments and the fragments are emulsified and then removed from the capsular bag by aspirating the fragments through a cannula formed as part of the phacoemulsification instrument. After the lens has been removed, the capsular bag must be cleaned in order to prepare it for the insertion of an IOL. In particular, epithelial, and cortical tissue fragments must be removed from the floor and perimeter of the capsular bag. Typically, an irrigation and aspiration tip is coupled to a hand piece, which may be the phacoemulsification hand piece, for removal of the fragments. The hand piece will typically have an irrigation and aspiration mode specifically for this task. U.S. Patent Application Publication No. US 2016/0374854 A1, hereby incorporated by reference in its entirety, discloses an irrigation and aspiration apparatus have any enlarged, ball-like tip, and a sheath portion that locks into a recess on the outer surface of the cannula.

Complete removal of the cortex is important for several reasons. If the cortex is not completely removed, it may cause post-operative inflammation and an increase in intraocular pressure. Incomplete removal of the cortex may also cause decentration or tilting of the IOL which, in turn, would cause a postoperative refractive error or induced astigmatism. This is especially important if the IOL is a multi-focal or toric type. Incomplete removal of the cortex may also result in the formation of after (secondary) cataract which would impair vision and require laser capsulotomy.

The present invention is directed to an irrigation and aspiration apparatus or assembly which has been specifically configured to promote safe and efficient irrigation and aspiration of the capsular bag attendant to eye surgery such as implantation of an intraocular lens.

### SUMMARY OF THE INVENTION

The present invention is directed to an improved surgical apparatus which is particularly suited for use with an associated irrigation and/or aspiration handpiece.

In accordance with one broad form of the present invention, the instrument apparatus includes a hollow cannula having a proximal end and a distal end, and defines an interior passage extending from the proximal end to the distal end. The interior passage defines a central cannula axis. The cannula includes at least one locking aperture extending transversely relative to the central cannula axis. The apparatus has a hollow tip with a base portion affixed within the distal end of the cannula. The base portion includes at least one projection extending within the at least one locking aperture of the cannula for maintaining the tip in position at the distal end of the cannula. The tip has an exposed end portion extending beyond the distal end of the cannula and defines at least one port in fluid communication with the interior passage defined by the cannula.

In one preferred form of the present invention, the at least one port defined by the exposed end portion of the tip extends transversely relative to the central cannula axis.

In another preferred form of the present invention, the at least one port has the form of a pair of opposing ports.

In yet another preferred form of the present invention, the at least one port has a stadium shape.

In yet another preferred form of the present invention, the cannula includes a pair of opposing locking apertures extending transversely relative to the central cannula axis and the base portion includes a pair of projections received therein.

According to one preferred form of the present invention, the at least one port and the at least one projection of the tip are aligned.

According to one preferred form of the present invention, the exposed end portion of the tip includes a flange surrounding a central tip axis.

In another preferred form of the present invention, the exposed end portion of the tip defines an outer tip surface, and the cannula defines an outer cannula surface which is coextensive with the outer tip surface.

In one preferred form of the present invention, the exposed end portion of the tip tapers radially inwardly to a rounded terminal end.

In another preferred form of the present invention, the exposed end portion of the tip includes a semi-circular terminal end.

According to one preferred form of the present invention, a proximal end of the cannula is provided with means for being removably attached to a handpiece.

According to one preferred form of the present invention, the apparatus is provided in combination with an infusion sleeve mounted around the cannula. The infusion sleeve includes a proximal end and a distal end terminating axially inwardly of the at least one port of the tip. The distal end of the infusion sleeve includes at least one infusion port. The instrument may be combined with a handpiece connected to a vacuum source and/or a fluid supply source.

In another preferred form of the present invention, the cannula is formed with at least one bend between the proximal and distal ends.

In one preferred form of the present invention, the tip is formed from an elastomer.

According to one preferred form of the present invention, the exposed end portion of the tip defines an outer tip surface that is roughened.

In still another form of the present invention, the tip is insert molded at the distal end of the cannula.

In still another form of the present invention, the port is elongate and extends transverse to the central axis of the tip.

In other broad forms, the tip may include one port for use in bimanual aspiration procedures and two or more ports for use in bimanual irrigation procedures.

### BRIEF DESCRIPTION OF THE DRAWINGS

In the accompanying drawings forming part of the specification, in which like numerals are employed to designate like parts throughout the same,

FIG. 1 is a right-side elevational view of a first illustrated embodiment of a surgical apparatus of the present invention for being attached to a handpiece that is connected to a vacuum source and, optionally, is connected to an irrigation fluid supply source;

FIG. 2 is a fragmentary, cross-sectional view of the distal portions of the instrument shown in FIG. 1, taken along a vertical plane extending through a central axis of the apparatus passage;

FIG. 3 is a left side elevational view of just the tip of FIG. 1 prior to assembly with the cannula of FIG. 1;

FIG. 4 is a top plan view of the tip of FIG. 3;

FIG. 5 is a cross-sectional view of the tip of FIG. 3, taken along the along a vertical plane extending through a central axis of the apparatus passage;

FIG. 6 is a right-side elevational view of a second illustrated embodiment of a surgical apparatus of the present invention for being attached to a handpiece that is connected to a vacuum source and, optionally, is connected to an irrigation fluid supply source;

FIG. 7 is another right-side elevational view of the apparatus of FIG. 6;

FIG. 8 is an isometric view, from below and the left, of the apparatus of FIG. 6;

FIG. 9 is a greatly enlarged, fragmentary, isometric view, from below and left-side, of only the operative distal portions of the instrument shown in FIG. 6;

FIG. 10 is a greatly enlarged, fragmentary, right-side elevational view of only the operative distal portions of the instrument shown in FIG. 6;

FIG. 11 is a right-side elevational view of a third illustrated embodiment of a surgical apparatus of the present invention for being attached to a handpiece that is connected to a vacuum source and, optionally, that is connected to an irrigation fluid supply source;

FIG. 12 is a fragmentary, cross-sectional view of the distal portions of the instrument shown in FIG. 11, taken along a vertical plane extending through a central axis of the apparatus passage;

FIG. 13 is a left side elevational view of just the tip of FIG. 11 prior to assembly with the cannula of FIG. 11;

FIG. 14 is a top plan view of the tip of FIG. 13;

FIG. 15 is a cross-sectional view of the tip of FIG. 13, taken along the along a vertical plane extending through a central axis of the apparatus passage;

FIG. 16 is a right-side elevational view of a fourth illustrated embodiment of a surgical apparatus of the present invention for being attached to a handpiece that is connected to a vacuum source and, optionally, is connected to an irrigation fluid supply source;

FIG. 17 is a greatly enlarged, fragmentary, right side elevational view, of only the operative distal portions of the instrument shown in FIG. 16;

FIG. 18 is another right-side elevational view of the apparatus of FIG. 16;

FIG. 19 is a greatly enlarged, fragmentary left-side elevational view of the apparatus of FIG. 16;

FIG. 19A is a top plan view of just the tip of FIG. 16 prior to assembly with the cannula of FIG. 16;

FIG. 19B is an isometric view of the tip of FIG. 19A;

FIG. 19C is a cross-sectional view of the tip of FIG. 19A, taken along the along a vertical plane extending through a central axis of the tip flow passage;

FIG. 19D is a right side elevational view of the tip of FIG. 19A, the left side elevational view being identical;

FIG. 19E is a right side elevational view of a single port version of the tip of FIG. 19A, the left side elevational view being identical;

FIG. 19F is a top plan view of the single port version of the tip of FIG. 19E;

FIG. 20 is a greatly enlarged, cross-sectional view of a fifth illustrated embodiment of a tip portion of an apparatus of the present invention assembled with an infusion sleeve, wherein FIG. 20 omits the cannula:

FIG. 21 is a right-side elevational view of the assembly shown in FIG. 20;

FIG. 22 is a greatly enlarged, fragmentary isometric view, from the right-side and below, of only the operative distal portions of the assembly in FIG. 21;

FIG. 23 is a diagrammatic view of the apparatus of FIG. 10 assembled with a handpiece, a vacuum supply source, an irrigation sleeve, and an irrigation supply source;

FIG. 24 is an isometric view of a sixth illustrated embodiment of a tip portion of an apparatus of the present invention for being assembled with a cannula, wherein FIG. 24 omits the cannula which may be connected to a handpiece that is in communication with a vacuum source and or irrigation fluid supply source;

FIG. 25 is a top plan view of the tip of FIG. 24;

FIG. 26 is a right side elevational view of the tip of FIG. 24;

FIG. 27 is a top plan view of the tip of FIG. 24, wherein FIG. 27 illustrates the internal structure of the tip;

FIG. 28 is a bottom plan view of the tip of FIG. 24;

FIG. 29 is a front elevational view the tip of FIG. 24;

FIG. 30 is a cross-sectional view of the tip of FIG. 24, taken along the along a vertical plane extending through a central axis of the tip flow passage;

FIG. 31 is a left side elevational view of the tip of FIG. 24;

FIG. 31A is a top plan view of a single port version of the tip of FIG. 24;

FIG. 31B is a right side elevational view of the single port version of the tip of FIG. 31A, the left side elevational view being identical;

FIG. 32 is a top plan view of a seventh illustrated embodiment of a tip portion of an apparatus of the present invention for being assembled with a cannula, wherein FIG. 32 omits the cannula which may be connected to a handpiece that is in communication with a vacuum source and or irrigation fluid supply source;

FIG. 33 is a left side elevational view of the tip of FIG. 32;

FIG. 34 is an isometric view of the tip of FIG. 32;

FIG. 35 is a top plan view of the tip of FIG. 32, wherein FIG. 35 illustrates the internal structure of the tip;

FIG. 36 is a bottom plan view of the tip of FIG. 32;

FIG. 37 is a cross-sectional view of the tip of FIG. 32, taken along the along a vertical plane extending through a central axis of the tip flow passage;

FIG. 37A is a top plan view of a single port version of the tip of FIG. 32;

FIG. 37B is a right side elevational view of the single port version of the tip of FIG. 37A, the left side elevational view being identical;

FIG. 38 is a top plan view of an eighth illustrated embodiment of a tip portion of an apparatus of the present invention for being assembled with a cannula, wherein FIG. 38 omits the cannula which may be connected to a handpiece that is in communication with a vacuum source and or irrigation fluid supply source;

FIG. 39 is a left side elevational view of the tip of FIG. 38;

FIG. 40 is an isometric view of the tip of FIG. 38;

FIG. 41 is a rear elevational view of the tip of FIG. 38;

FIG. 42 is a top pan view of the tip of FIG. 38, and FIG. 42 illustrates the internal structure of the tip;

FIG. 43 is another top plan view of the tip of FIG. 38;

FIG. 44 is a front elevational view the tip of FIG. 38;

FIG. 45 is a cross-sectional view of the tip of FIG. 38, taken along the along a vertical plane extending through a central axis of the tip flow passage;

FIG. 46 is a right side elevational view of the tip of FIG. 38;

FIG. 47 is an isometric view of a ninth illustrated embodiment of a tip portion of an apparatus of the present invention for being assembled with a cannula, wherein FIG. 47 omits the cannula which may be connected to a handpiece that is in communication with a vacuum source and or irrigation fluid supply source;

FIG. 48 is a rear elevational view the tip of FIG. 47;

FIG. 49 is a top plan view of a tenth illustrated embodiment of a tip portion of an apparatus of the present invention for being assembled with a cannula, wherein FIG. 49 omits the cannula which may be connected to a handpiece that is in communication with a vacuum source and or irrigation fluid supply source;

FIG. 50 is an isometric view the tip of FIG. 49;

FIG. 51 is a right side elevational view of the tip of FIG. 49;

FIG. 52 is a top plan view of the tip of FIG. 49, and FIG. 52 illustrates the internal structure of the tip;

FIG. 53 is a bottom plan view of the tip of FIG. 49;

FIG. 54 is a cross-sectional view of the tip of FIG. 49, taken along the along a vertical plane extending through a central axis of the tip flow passage;

FIG. 54A is a top plan view of a single port version of the tip of FIG. 49; and

FIG. 54B is a left side elevational view of the single port version of the tip of FIG. 54A, the right side elevational view being identical.

### DETAILED DESCRIPTION OF THE PREFERRED EMBODIMENTS

While the present invention is susceptible of embodiment in various forms, there are shown in the drawings and will hereinafter be described the presently preferred embodiments, with the understanding that the present disclosure should be considered as an exemplification of the invention, and is not intended to limit the broadest forms of the invention to only the specific embodiments illustrated.

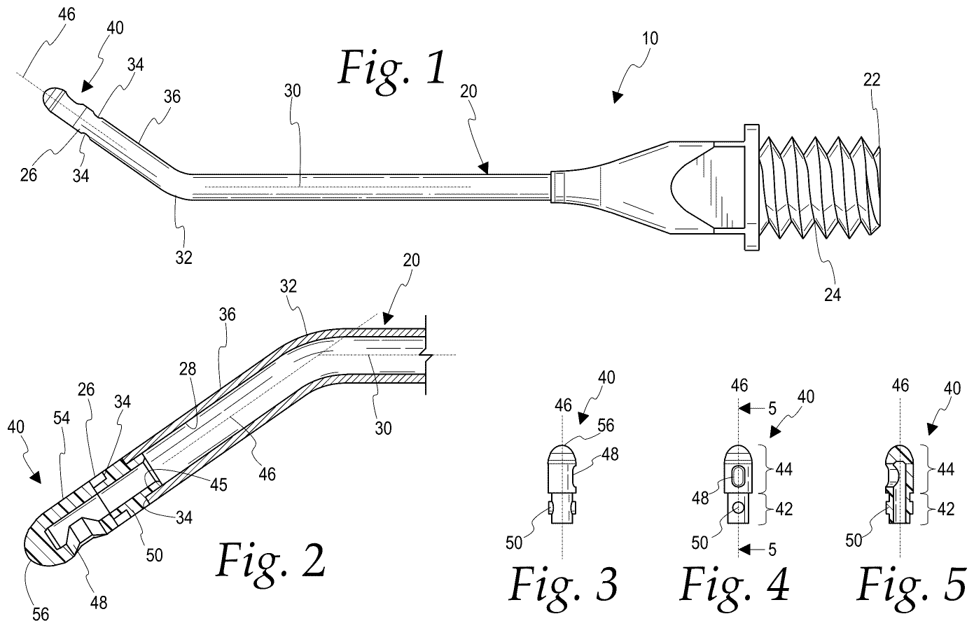

A first illustrated embodiment of a surgical apparatus or irrigation/aspiration instrument 10 according to the present invention is shown in FIGS. 1-5, wherein the instrument 10 includes a hollow cannula or shaft 20 having a proximal end 22 with means 24 in the form of threads for being connected to mating threads in an irrigation handpiece or irrigation/aspiration handpiece and a distal end 26 spaced from the proximal end 22. The cannula 20 defines an internal passage 28 (FIG. 2), through which aspiration or irrigation is effected during surgery when the proximal end 22 is connected to a vacuum source or a fluid supply source via the handpiece (FIG. 29). It will be understood that the proximal end 22 of the instruments disclosed herein may have a variety of means or structures suited for being attached to a variety of handpieces, such as by mating screw threads, clamps, locks, friction fitting, etc. Suitable proximal end structures of instruments are described in U.S. Pat. No. 8,764,782, the entirety of which is incorporated herein by reference.

With reference to FIG. 2, the cannula 20 defines a central cannula axis or centerline 30 that extends from the proximal end 22 to the distal end 26. The cannula 20 includes a bend 32 between the proximal end 22 to the distal end 26, preferably about 35 degrees relative to the axis 30. The specific configuration of the cannula 20 can be varied depending upon intended use or uses, and may be bent in other configurations, or may be straight. Other bent configurations are contemplated. The distal end 26 of the cannula 20 includes a pair of opposing, circular locking apertures 34 located on opposite sides of the central axis 30. Each one of the locking apertures 34 extends through an outer surface 36 of the cannula 20 generally transverse to the portion of the axis 30 distal of the bend 32.

Still referring to FIG. 2, the present instrument 10 further comprises a hollow tip 40 attached to the distal end 26 of the cannula 20, opposite of the proximal end 22. Importantly, the tip 40 is formed from a polymer, preferably an elastomer such as silicone, which is substantially softer and less rigid than the material of the cannula 20 (which may be metal or a rigid or hard polymer) to permit the instrument 10 to safely contact the lens capsule during use.

With reference FIG. 5, the tip 40 includes a base portion 42 for being received within the open distal end 26 of the cannula 20 and an exposed end portion 44 that extends distally outwardly of the distal end 26 of the cannula 20 (as illustrated in FIG. 2). The tip 40 is hollow, including a tip passage 45 defining a central tip axis 46 and defines a port 48 which fluidly connects to the passage 28 of the cannula 20 to facilitate aspiration of fluids and tissues through the instrument 10 when subjected to a vacuum or flow of irrigation fluid when connected to a fluid supply source. The port 48 is elongate, having an obround or stadium shape (as can be seen in FIG. 4).

As can be seen in FIGS. 3-5, the base portion 42 of the tip 40 includes a pair of opposite, retention projections 50 extending transversely to the tip axis 46. Each one of the projections 50 is received in one of the locking apertures 34 of the cannula 20 for securing the tip 40 at the distal end 26. The projections 50 and the port 48 are aligned with, and symmetric about, a shared plane extending through the tip axis 46 (as seen in FIG. 4).

Referring next to FIG. 2, the exposed end portion 44 defines an outer surface 54 that is substantially coextensive with the outer surface 36 of the cannula 20 at the distal end 26. The exposed end portion 44 has a generally bullet shape which tapers radially inwardly (toward the tip axis 46) to a rounded distal point 56. The taper is preferably about 45 degrees relative to the tip axis 46. The arrangement of the instrument 10 illustrated in FIGS. 1-5 is ideally suited as an aspiration instrument.

A desirable feature of the present instrument 10 is the tip 40 is more securely locked with the cannula 20 by insert molding and may exhibit improved durability of the tip 40 compared to prior art instruments. The bullet shaped, tapered shape of the exposed end portion 44 may further enter through incisions more easily than prior art instruments.

It is contemplated that the outer surface 54 of the tip 20 may have a smooth or a roughened surface for polishing of the lens capsule.

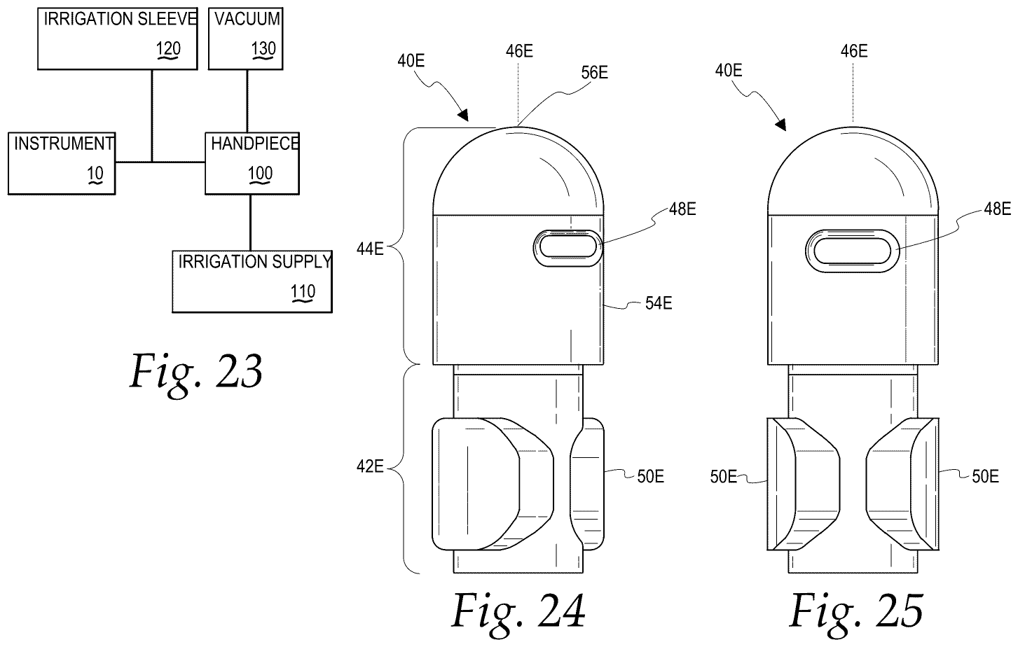

With reference to FIG. 23, the instrument 10 may be connected to a handpiece 100 and an irrigation fluid supply 110 (with or without a sleeve 120) and/or a vacuum supply 130. Accordingly, the instrument 10 may function solely as an aspiration device, solely as an irrigation device, or a function as an aspiration and irrigation device when coupled with a sleeve 120. The sleeve 120 may have any form. One such form is the elastomeric sleeve 120 shown in FIGS. 12-14 which is coaxially fitted around the cannula 20 to supply fluid proximate to the distal end 26 thereof.

The inventor has found that the instrument 10 has an advantageous tip 40 with a smaller overall size compared to prior art irrigation and/or aspiration instruments for improved passage through the corneal incision. The port 48 has a greater surface area compared to prior art instruments for more effective aspiration. The elongate port 48 allows for increased flow, while the narrow width of the port may improve irrigation and/or aspiration flow speed through the port 48. The tip 40 may advantageously be formed in either soft thermoplastic or hard thermoplastic materials, depending on the application. The instrument 10 may be used for bimanual and coaxial irrigation and/or aspiration techniques for cataract procedures.

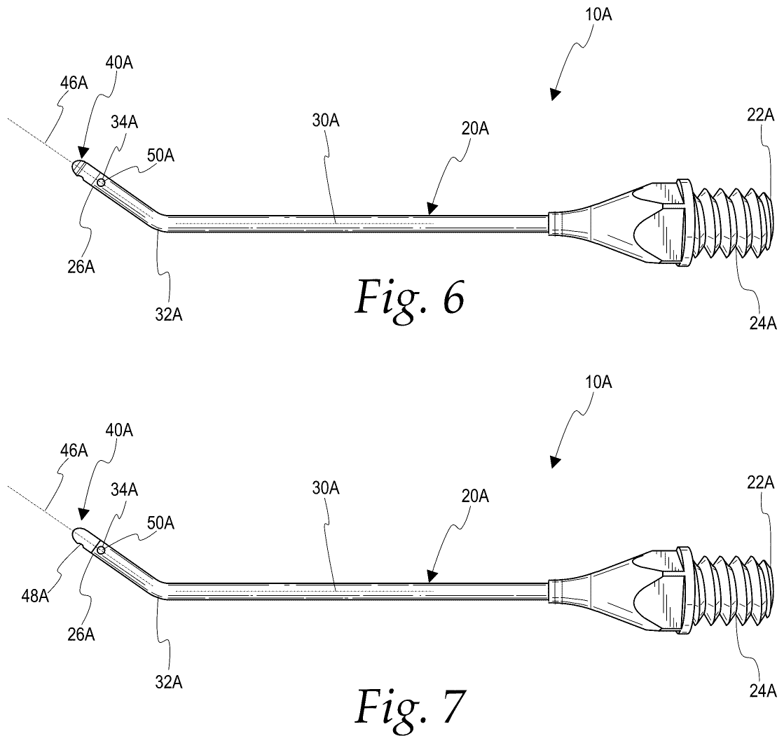

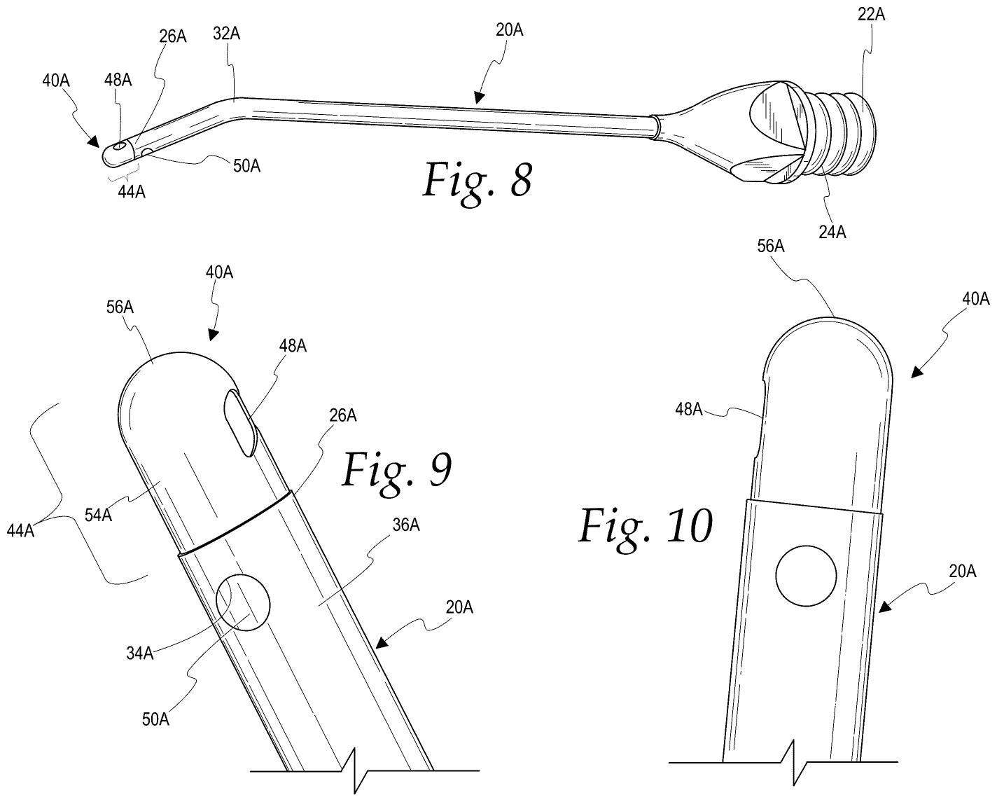

A second embodiment of an instrument according to the present invention is shown in FIGS. 6-10, designated by the numeral 10A, and functions similarly to the first illustrated embodiment of the instrument 10 as previously described above and illustrated in FIGS. 1-5. The numbered features of the second embodiment of the instrument 10A illustrated in FIGS. 6-10 are analogous to features of the first embodiment of the instrument 10 that share the same number, but without the suffix “A”. The second embodiment of the surgical instrument 10A differs from the aforementioned first illustrated embodiment in that the second embodiment includes a tip 20A having an exposed end portion 44A which has a more blunted, semi-circular configuration terminating at a distal point 56A.

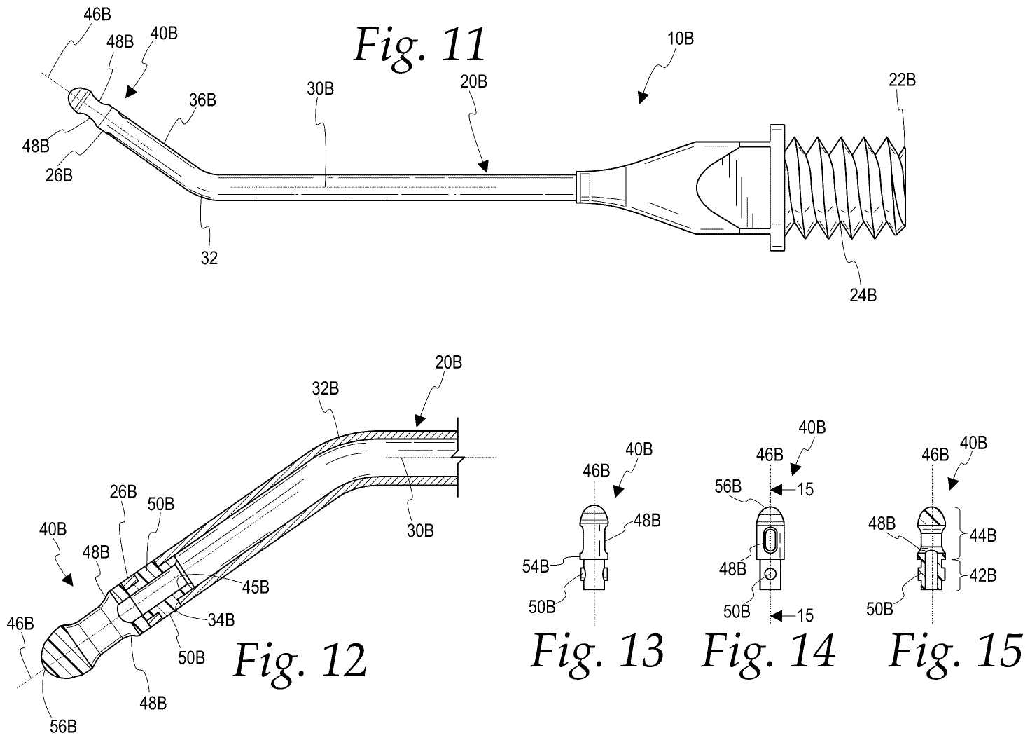

A third embodiment of a surgical instrument according to the present invention is shown in FIGS. 11-15, designated by the numeral 10B, and functions similarly to the first illustrated embodiment of the instrument 10 as previously described above and illustrated in FIGS. 1-5. The numbered features of the third embodiment of the instrument illustrated in FIGS. 11-15 are analogous to features of the first embodiment of the instrument that share the same number (without the suffix “B”). The third embodiment of the surgical instrument 10B differs from the aforementioned first illustrated embodiment in that the instrument 10B includes a tip 40B having an exposed end portion 44B with a pair of opposite, ports 48B which are in fluid communication with the passage 28B of the cannula 20B. The arrangement of the instrument 10B illustrated in FIGS. 11-15 is ideally suited as an irrigation instrument.

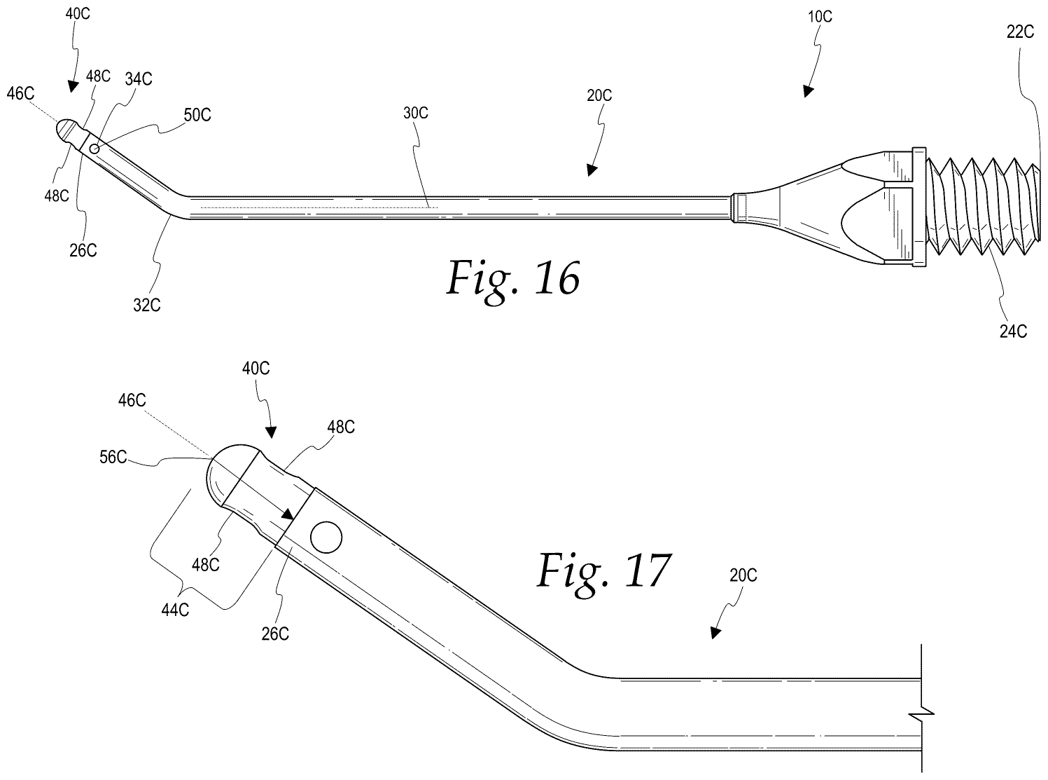

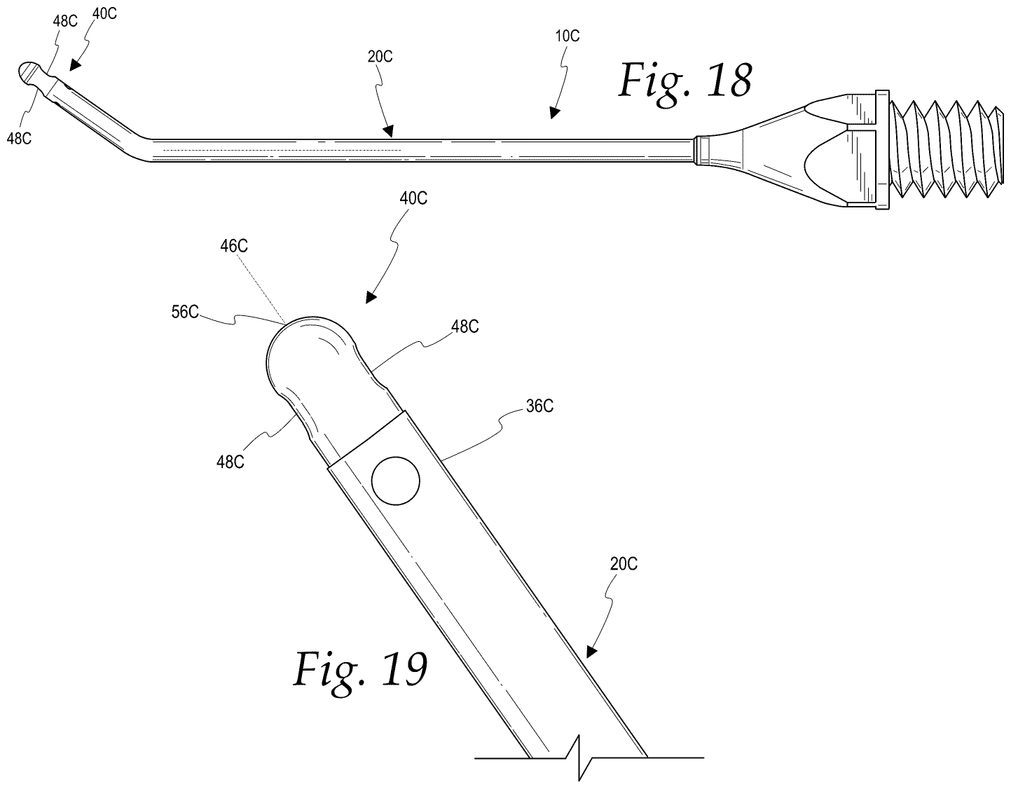

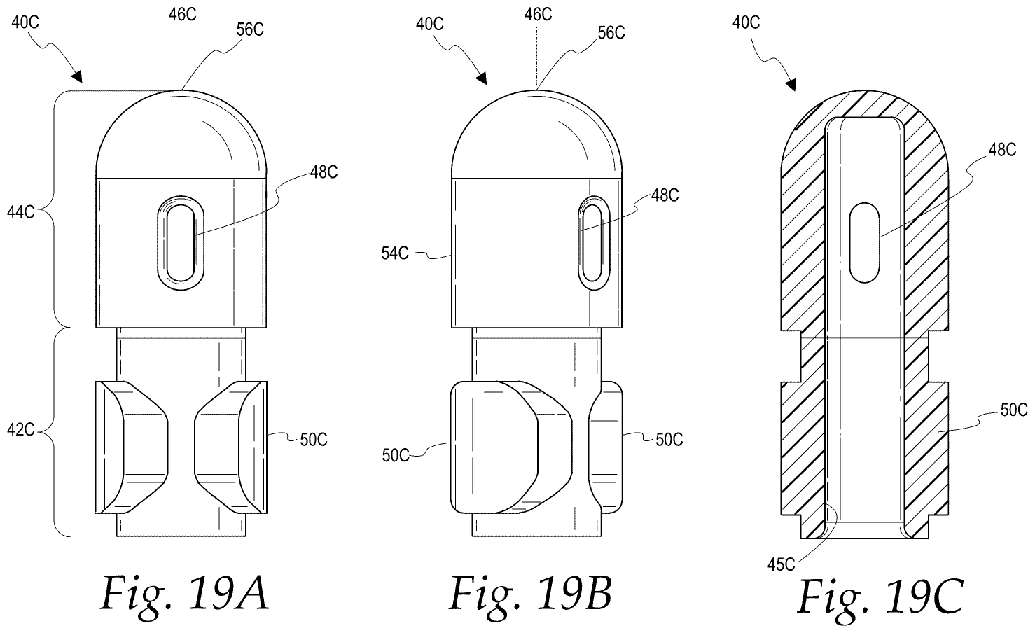

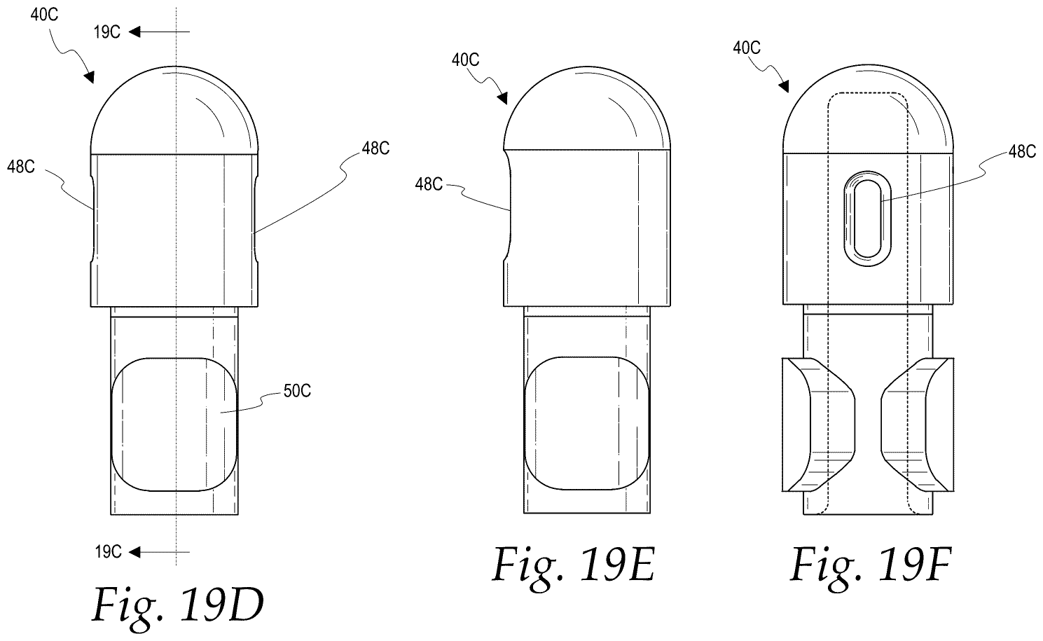

A fourth embodiment of a surgical instrument according to the present invention is shown in FIGS. 16-19D, designated by the numeral 10C, and functions similarly to the third illustrated embodiment of the instrument 10B as previously described above and illustrated in FIGS. 11-15. The numbered features of the fourth embodiment of the instrument illustrated in FIGS. 16-19D are analogous to features of the third embodiment of the instrument that share the same number (without the suffix “C”). The fourth embodiment of the surgical instrument 10C differs from the aforementioned third illustrated embodiment in that the instrument 10C includes a tip 20C having an exposed end portion 44C which has a more blunted, semi-circular configuration terminating at a distal point 56C. With reference to FIGS. 19A-19D, the tip 40C may further include rounded, square shaped projections 50C and a narrowed pair of elongate ports 48C that are aligned with the central axis 46C of the tip 40C. The ports 48C have a preferred axial length of about 0.3 mm and width (normal to the axis 46C) of about 0.1 mm. The inventors have found that the tip 40C is ideally suited for use as a bimanual irrigation instrument when coupled with a cannula. The elongate ports 48C allow for increased flow through the tip 40E, while the narrow width of each port 48C may improve irrigation and/or aspiration flow speed through the port 48. With reference to FIGS. 19E and 19F, it is contemplated that the tip 40C may be modified to have only a single elongate, vertical axis aligned port 48C as described immediately above for use as a bimanual aspiration instrument when coupled with a cannula.

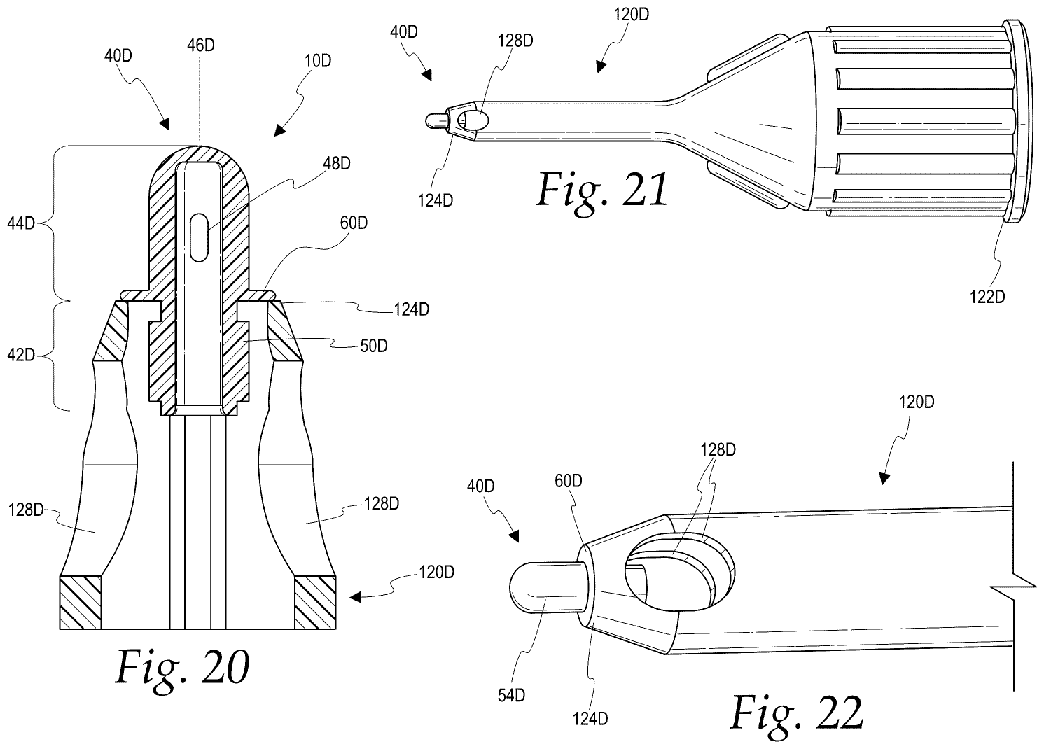

A fifth embodiment of a surgical instrument according to the present invention is shown in FIGS. 20-22, designated by the numeral 10D, and functions similarly to the first illustrated embodiment of the instrument 10 as previously described above and illustrated in FIGS. 1-5. The numbered features of the fifth embodiment of the instrument illustrated in FIGS. 20-22 are analogous to features of the first embodiment of the instrument that share the same number (without the suffix “D”). The fifth embodiment of the surgical instrument 10D differs from the aforementioned first illustrated embodiment in that the instrument 10D includes a tip 40D having an exposed end portion 44D with a flange 60D surrounding the tip axis 56D. The flange 60D is for blocking or at least inhibiting flow of an irrigation fluid from the distal end of an infusion sleeve 120D. The sleeve 120D is hollow and includes a proximal end 122D for receiving an infusion fluid, a distal end 124D for abutting the flange 60D, and a pair of infusion fluid ports 128D which are in fluid communication with the interior of the hollow sleeve 120D. It will be noted that the cannula of the instrument 10D is omitted in FIGS. 20-22 for improved visibility of the tip 40D and sleeve 120D, but it will be understood that the cannula would be identical to that described immediately above with respect to the first embodiment of the instrument 10.

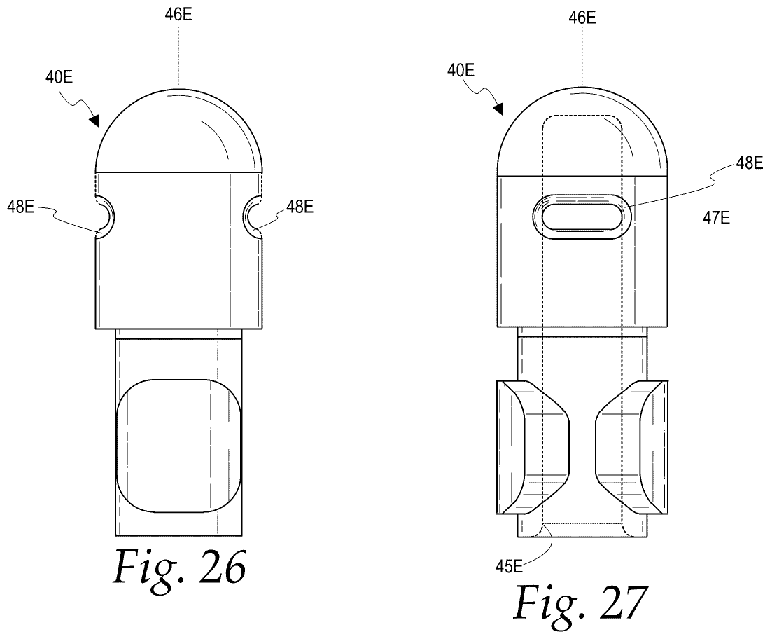

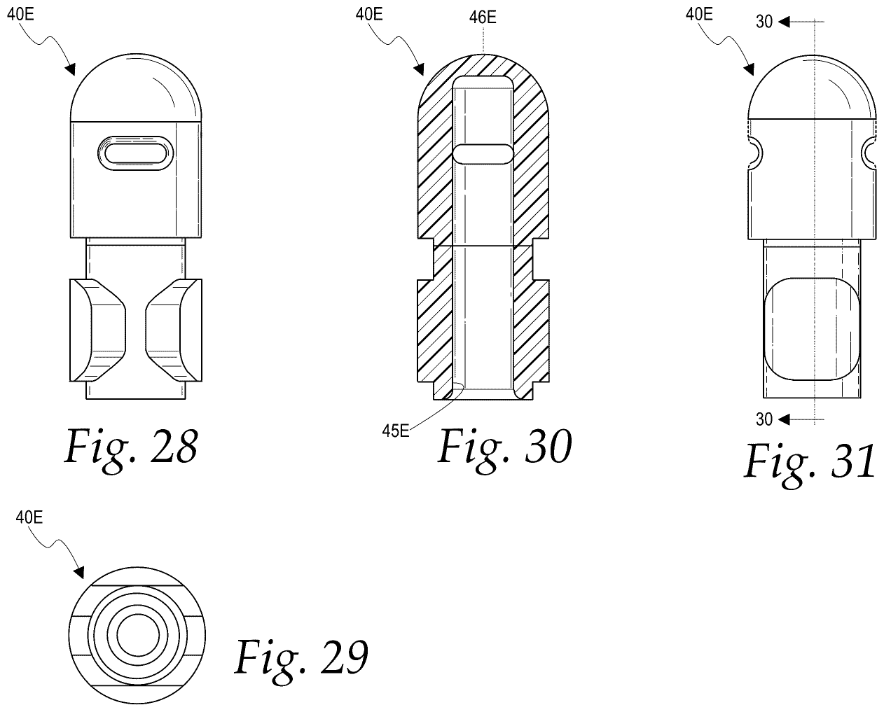

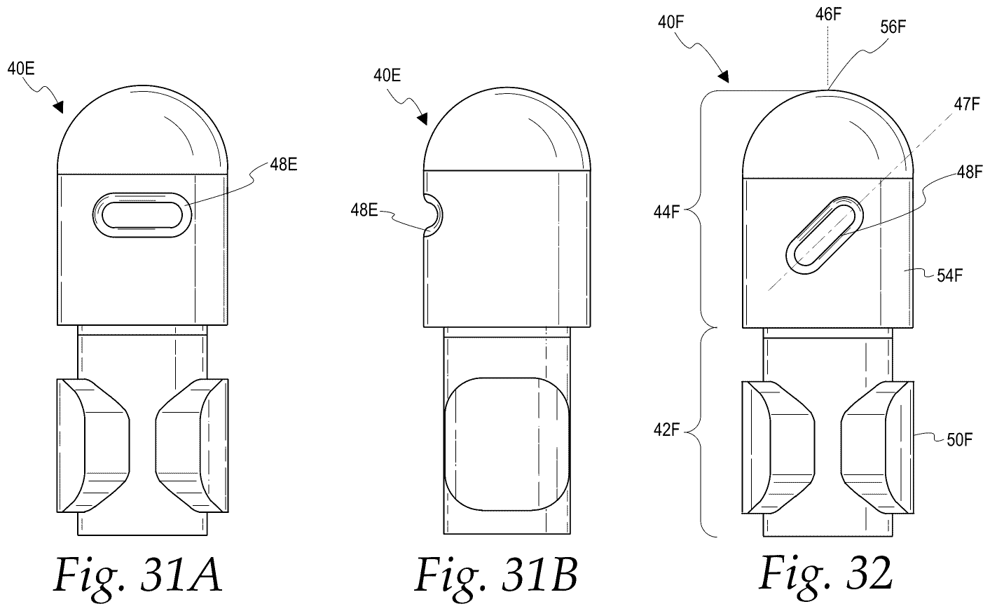

A sixth embodiment of a tip for a surgical instrument according to the present invention is shown in FIGS. 24-31, designated by the numeral 40E, and functions similarly to the tip 40C of the fourth illustrated embodiment of the instrument 10C as previously described above and illustrated in FIGS. 16-19D. The numbered features of the sixth embodiment of the instrument illustrated in FIGS. 24-31 are analogous to features of the fourth embodiment of the instrument that share the same number (without the suffix “E”). The sixth embodiment of the tip 40E differs from the aforementioned fourth illustrated embodiment in that the tip 40E has a narrowed pair of elongate ports 48E that are aligned relative to a transverse axis 47E, which is perpendicular or normal relative to the central axis 46E of the tip 40E. The ports 48E have a preferred axial length of about 0.3 mm and width (normal to the axis 46E) of about 0.1 mm. The inventors have found that the tip 40E is ideally suited for use as a bimanual irrigation instrument when coupled with a cannula. The elongate ports 48E allow for increased flow through the tip 40E, while the narrow width of the port may increase irrigation and/or aspiration flow speed through the ports 48E. With reference to FIGS. 31A and 31B, it is contemplated that the tip 40E may be modified to have only a single elongate port 48E as described immediately above for use as a bimanual aspiration instrument when coupled with a cannula.

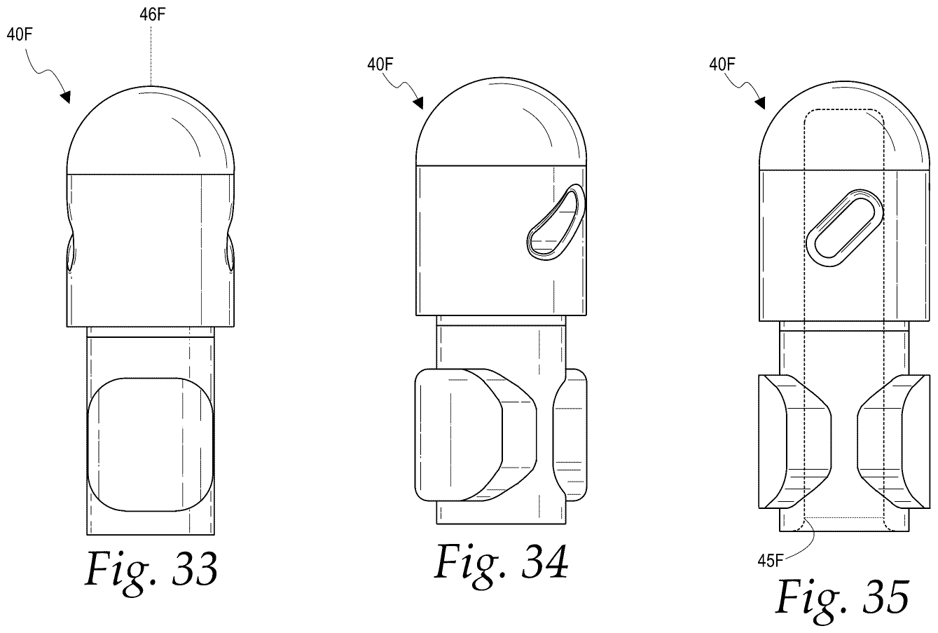

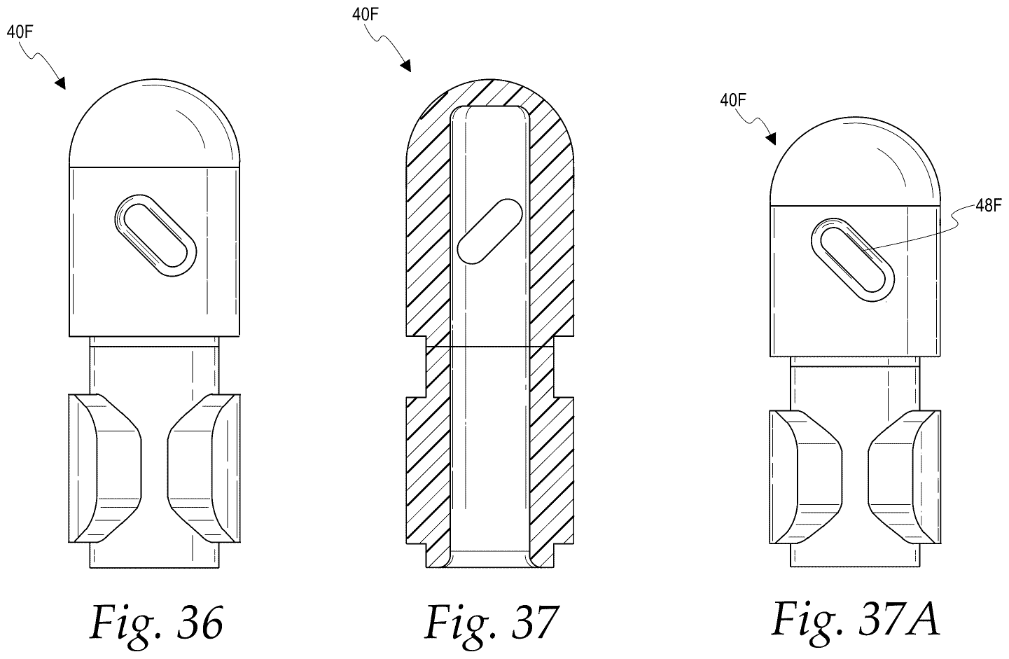

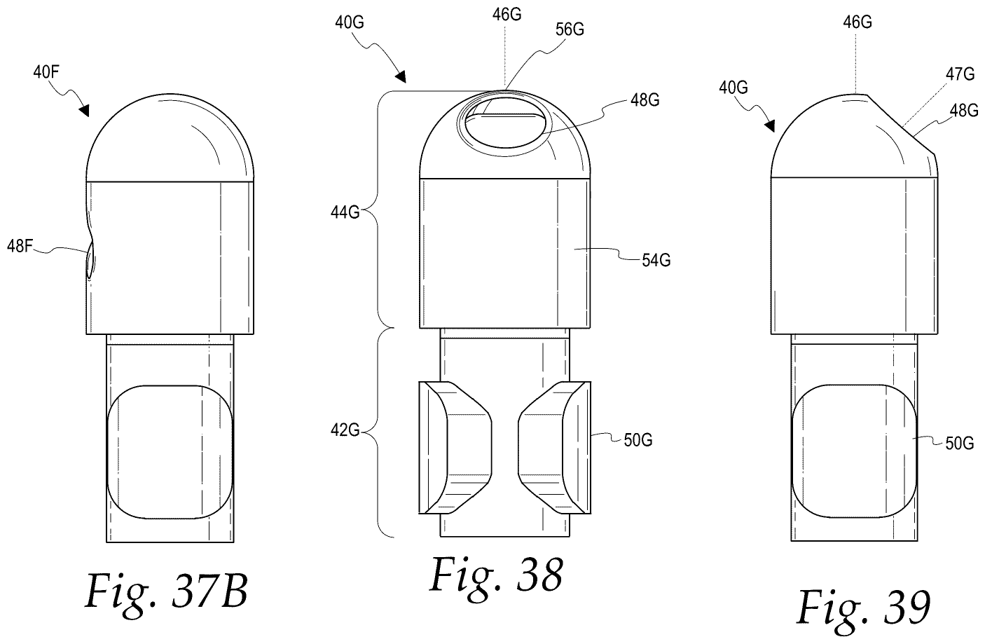

A seventh embodiment of a tip portion of a surgical instrument according to the present invention is shown in FIGS. 32-37, designated by the numeral 40F, and functions similarly to the tip 40E of the sixth illustrated embodiment of the instrument as previously described above and illustrated in FIGS. 24-31. The numbered features of the seventh embodiment of the instrument illustrated in FIGS. 32-37 are analogous to features of the sixth embodiment of the instrument that share the same number (without the suffix “F”). The seventh embodiment of the tip 40F of the surgical instrument differs in that the instrument it includes a narrowed pair of elongate ports 48F that are aligned relative to a transverse axis 47F, which is angled about 45 degrees relative to the central axis 46F of the tip 40F. The ports 48F have a preferred axial length of about 0.3 mm and width (normal to the axis 47F) of about 0.1 mm. The inventors have found that the tip 40F is ideally suited for use as a bimanual irrigation instrument when coupled with a cannula. With reference to FIGS. 37A and 37B, it is contemplated that the tip 40F may be modified to have only a single elongate, transverse, or angled port 48E as described for use as a bimanual aspiration instrument when coupled with a cannula.

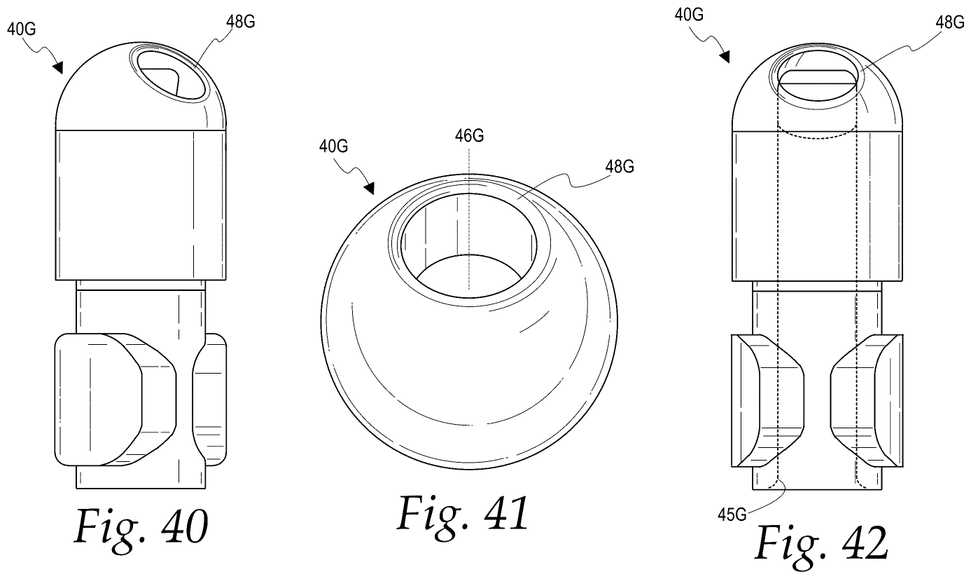

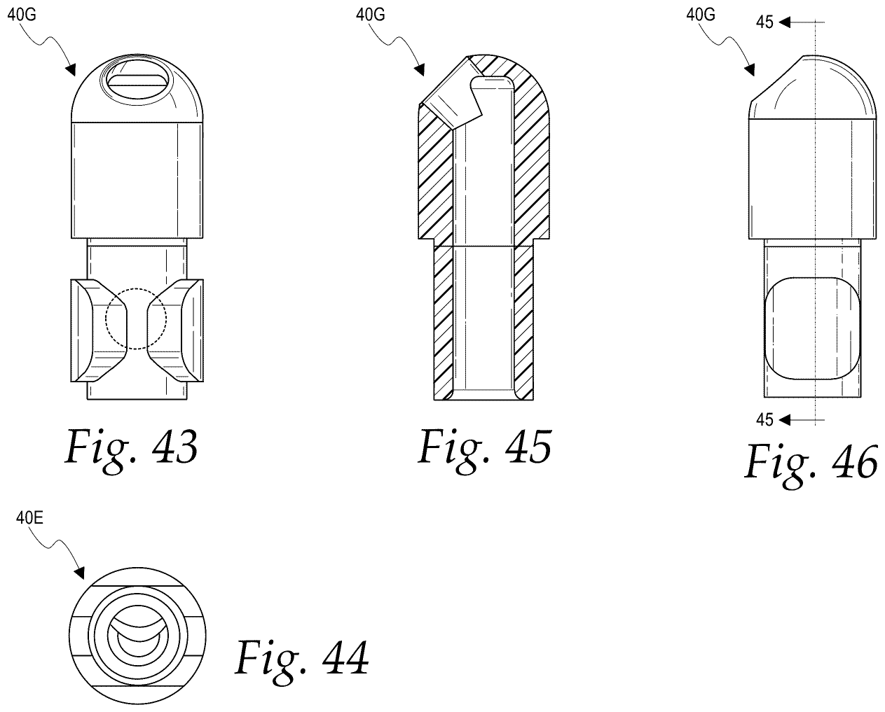

An eighth embodiment of a tip portion of a surgical instrument according to the present invention is shown in FIGS. 38-46, designated by the numeral 40G, and functions similarly to the tip 40 of the first illustrated embodiment of the instrument as previously described and illustrated in FIGS. 1-5. The numbered features of the eighth embodiment of the instrument illustrated in FIGS. 38-46 are analogous to features of the first embodiment of the instrument that share the same number (without the suffix “G”). The eighth embodiment of the tip 40G of the surgical instrument differs in that the instrument it includes a single, widened circular port 48G that is aligned relative to a transverse axis 47G, which is angled about 45 degrees relative to the central axis 46G of the tip 40G. The port 48G has a preferred diameter of about 0.3 mm. The port 48G is located about 90 degrees around the circumference of outer surface 54G of the tip 40G relative to the locking projections 50G that are received within the cannula (not illustrated). The inventors have found that the tip 40G is ideally suited for use as a bimanual aspiration instrument when coupled with a cannula. The flexible tip 40G may advantageously be used in a side-to-side motion of just the flexible tip 40G without appreciable movement of the handle or handpiece, and/or cannula of the instrument upon which the tip 40G is installed, thus avoiding negatively stretching the incision in the cornea through which the instrument cannula extends. The placement of the port 48 at a transverse angle relative to the central axis 46G allows removal of the cortex in a 360 degree swath.

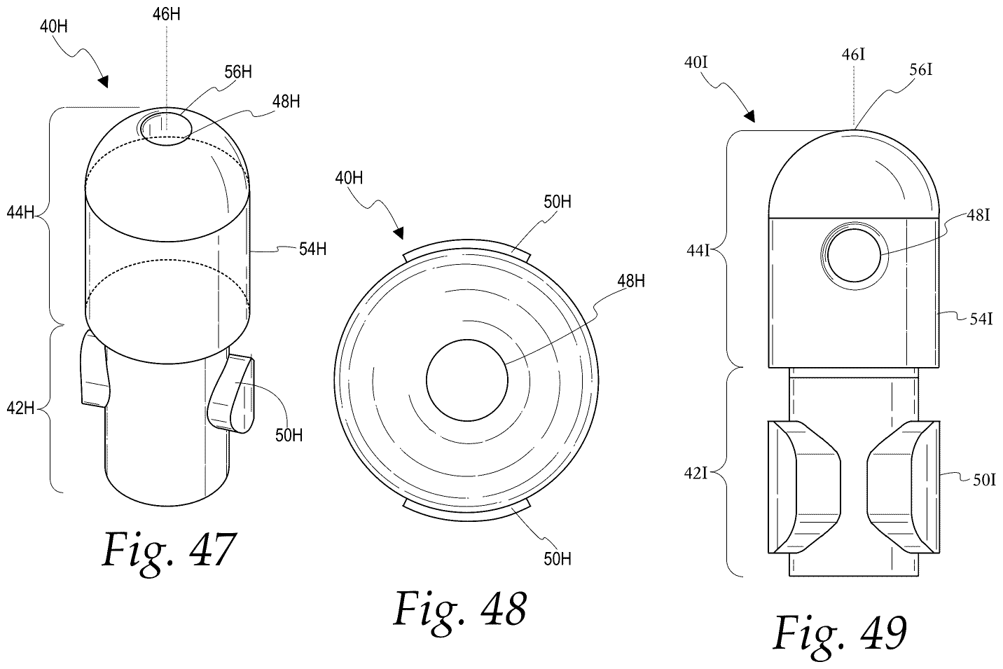

A ninth embodiment of a tip portion of a surgical instrument according to the present invention is shown in FIGS. 47-48, designated by the numeral 40H, and functions similarly to the tip 40 of the first illustrated embodiment of the instrument as previously described and illustrated in FIGS. 1-5. The numbered features of the ninth embodiment of the instrument illustrated in FIGS. 47-48 are analogous to features of the first embodiment of the instrument that share the same number (without the suffix “H”). The ninth embodiment of the tip 40H of the surgical instrument differs in that the instrument it includes a single, narrow circular port 48H that is aligned with the vertical, central axis 46H of the tip 40H. The port 48H has a preferred diameter of about 0.2 mm. The port 48H is located at the distal point 56H of outer surface 54H of the tip 40H. The inventors have found that the tip 40H is ideally suited for use as a bimanual aspiration instrument when coupled with a cannula. The location of the port 48H oriented along the central axis 46H permits the tip 40H to only remove cortex in the region surrounding, or proximate to, the central axis 46H.

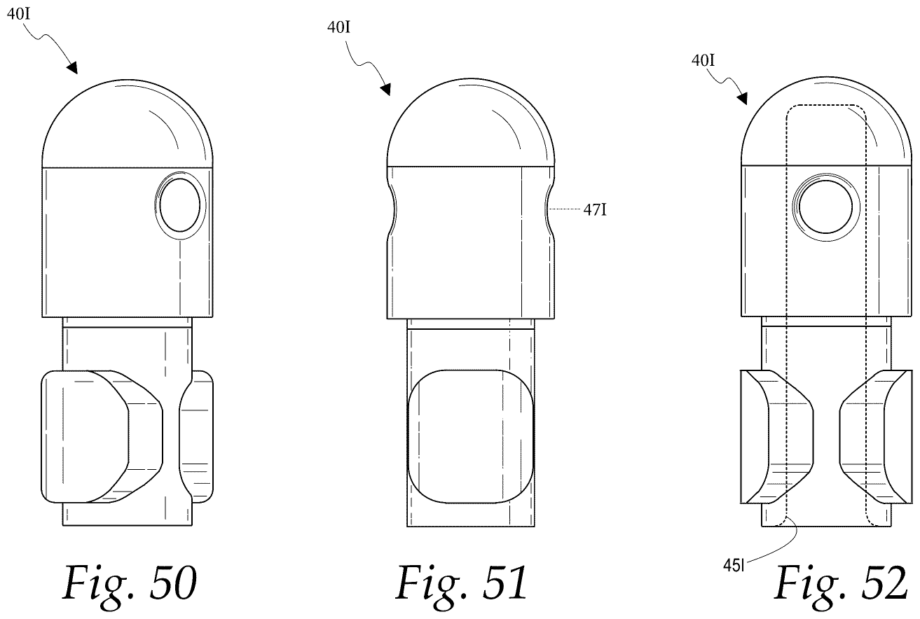

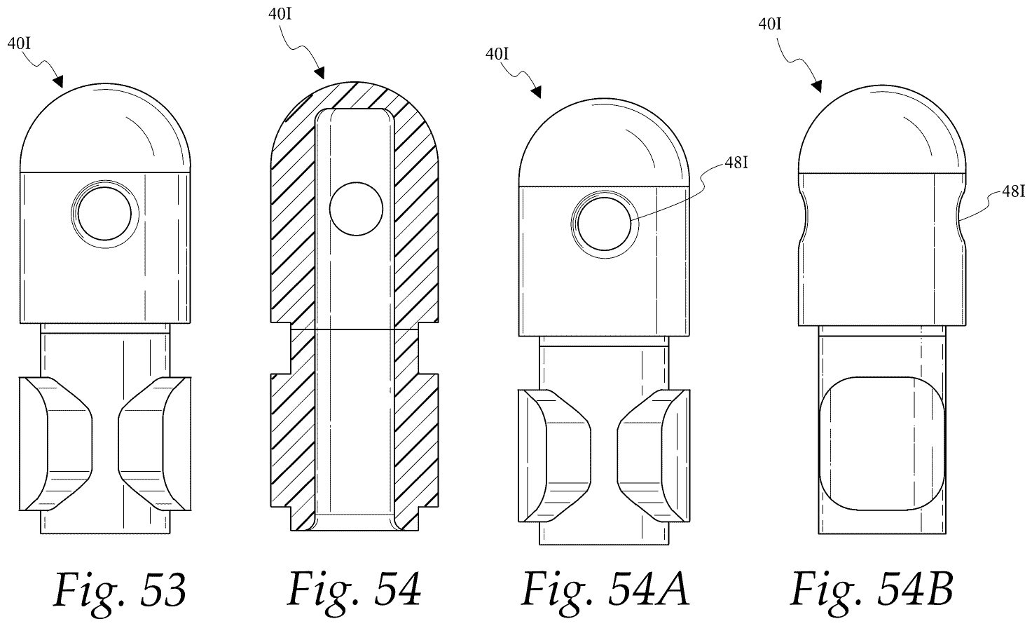

A tenth embodiment of a tip portion of a surgical instrument according to the present invention is shown in FIGS. 49-54, designated by the numeral 40I, and functions similarly to the tip 40C of the fourth illustrated embodiment of the instrument as previously described above and illustrated in FIGS. 17-19. The numbered features of the tenth embodiment of the instrument illustrated in FIGS. 49-54 are analogous to features of the tenth embodiment of the instrument that share the same number (without the suffix “I”). The tenth embodiment of the tip 40I of the surgical instrument differs in that the instrument it includes a pair of opposite, circular ports 48I that are aligned relative to a transverse axis 47I, which is angled about 90 degrees relative to the central axis 46I of the tip 40I. The ports 48I have a preferred diameter of about 0.2 mm. The inventors have found that the tip 40I is ideally suited for use as a bimanual irrigation instrument when coupled with a cannula. With reference to FIGS. 54A and 54B, it is contemplated that the tip 40I may be modified to have only a single circular port 48I as described above for use as a bimanual aspiration instrument when coupled with a cannula.

From the foregoing, it will be observed that numerous modifications and variations can be effected without departing from the true spirit and scope of the novel concept of the present invention. It is to be understood that no limitation of the broadest concepts with respect to the specific embodiments illustrated herein is intended or should be inferred. The disclosure is intended to cover, by the appended claims, all such modifications as fall within the scope of the claims.

1. A surgical apparatus, said apparatus comprising:
a hollow cannula having a proximal end, a distal end, and defining an interior passage extending from said proximal end to said distal end, said interior passage defining a central cannula axis, said cannula including at least one locking aperture extending transversely relative to said central cannula axis; and
a hollow tip having a base portion affixed within the distal end of said cannula, said base portion including at least one projection extending within said at least one locking aperture of said cannula for maintaining said tip in position at said distal end of said cannula, said tip having an exposed end portion extending beyond said distal end of said cannula and defining at least one port in fluid communication with the interior passage defined by said cannula.

2. The surgical apparatus in accordance with claim 1, wherein said at least one port defined by said exposed end portion of said tip extends transversely relative to said central cannula axis.

3. The surgical apparatus in accordance with claim 1, wherein said at least one port has the form of a pair of opposing ports.

4. The surgical apparatus in accordance with claim 1, wherein said at least one port has a stadium shape.

5. The surgical apparatus in accordance with claim 1, wherein said cannula includes a pair of opposing locking apertures extending transversely relative to said central cannula axis and said base portion includes a pair of projections received therein.

6. The surgical apparatus in accordance with claim 1, wherein said at least one port and said at least one projection of said tip are aligned.

7. The surgical apparatus in accordance with claim 1, wherein said exposed end portion of said tip includes a flange surrounding a central tip axis.

8. The surgical apparatus in accordance with claim 1, wherein said exposed end portion of said tip defines an outer tip surface, and said cannula defines an outer cannula surface which is coextensive with said outer tip surface.

9. The surgical apparatus in accordance with claim 1, wherein said exposed end portion of said tip tapers radially inwardly to a rounded terminal end.

10. The surgical apparatus in accordance with claim 1, wherein said exposed end portion of said tip includes a semi-circular terminal end.

11. The surgical apparatus in accordance with claim 1 in combination with an infusion sleeve mounted around said cannula, said infusion sleeve having a proximal end and a distal end terminating axially inwardly of said at least one port of said tip, said distal end of said infusion sleeve including at least one infusion port.

12. The surgical apparatus in accordance with claim 1 wherein said cannula is formed with at least one bend between said proximal end and said distal end.

13. The surgical apparatus in accordance with claim 1 wherein said tip is formed from an elastomer.

14. The surgical apparatus in accordance with claim 1 wherein said exposed end portion of said tip defines an outer tip surface that is roughened.

15. The surgical apparatus in accordance with claim 1 wherein said tip is insert molded at said distal end of said cannula.

16. The surgical apparatus in accordance with claim 1 in combination with a handpiece connected to a vacuum source.

17. The surgical apparatus in accordance with claim 1 in combination with a handpiece connected to an infusion fluid supply source.

18.-34. (canceled)
